(19) United States
(12) Patent Application Publication
(10) Pub. No.: US 2008/0022945 A1
(43) Pub. Date: Jan. 31, 2008

(54) Leg bandages for animals

(76) Inventors: Jeffeny K. Hughes, Long Beach, CA (US); Joshua P. Hughes, Long Beach, CA (US)

Correspondence Address: Jeffeny K. Hughes, 1342 Bennett Ave, Long Beach, CA, 90804, US

(21) Appl. No.: 11/880,053

(22) Filed: Jul. 19, 2007

Related U.S. Application Data
(60) Provisional application No. 60/834,218, filed on Jul. 28, 2006.

(51) Int. Cl. A01K 29/00; A61B 19/12; A61F 13/06

(52) U.S. Cl. 119/850; 128/898; 602/63

(57) ABSTRACT

A bandage system for an animal that has at least two legs. The system comprises a first tubular member adapted to fit over one of the animal's legs, a second tubular member adapted to fit over another of the animal's legs. A support member is connected to the first tubular member and to the second tubular member. The support member is adapted to fit over the animal's back.

### CROSS-REFERENCE TO RELATED APPLICATIONS

This application claims the benefit of U.S. Provisional Patent Application No. 60/834,218 filed Jul. 28, 2006 by Jeffeny K. Scott and Joshua P. Hughes and titled “Leg Bandages for Animals.” U.S. Provisional Patent Application No. 60/834,218 filed Jul. 28, 2006 by Jeffeny K. Scott and Joshua P. Hughes and titled “Leg Bandages for Animals” is incorporated herein by this reference.

### FIELD OF ENDEAVOR

The present invention relates to injuries to animals and more particularly to leg bandages for animals.

### STATE OF TECHNOLOGY

U.S. Pat. No. 3,895,628 issued Jul. 22, 1975 to Georga Ann Adair for a body encompassing bandage protector for dogs and other such animals provides the following state of technology information: “Dogs and other animals have a tendency to bite and pull bandage coverings from body wounds. When the wound, whether it be a result of surgery or an accident, is unprotected by a bandage covering the healing process is likely to be slowed and the open wound is susceptible to infection. If the wound is infected the life of the animal is often placed in jeopardy and the recovery period is prolonged. In the past, to prevent the dog or animal from biting and pulling the bandage from the wound, it has been common practice, especially among veterinarians, to place an open bottom bucket type structure around the dog's neck and head. While this is a cumbersome and awkward approach, it nevertheless has been used and because of the seriousness of the problem has been to a limited extent successful. But besides being awkward and cumbersome, the open bottom bucket often restricts the mobility of the animal and seriously hampers the animal's ability to become comfortable. This is especially true when the animal attempts to rest or sleep by laying on his stomach or side.”

United States Published Patent Application No. 2004/0031448 by Diana M. Trepanier published Feb. 19, 2004 for a post-surgical care bandage cover for pets provides the following state of technology information: “It would be desirable, to provide a cover for a pet which is easily mounted and removed and which can effectively cover a dressed or undressed wound, protecting the area from constant washing, and keeping the wound clean, allowing air flow but excluding dust and dirt. It would also be desirable to provide such a cover which provides easy access to the wound for inspection by a veterinarian without entirely removing the cover, a task which may be laborious and time consuming, depending on the size and disposition of the pet, while assuring that the pet cannot remove the cover for access to the wound. It would also be desirable to provide a cover which is durable, machine washable, stylish, amenable to decoration, available in a variety of colors and sizes while remaining affordable for any dog owner.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a bandage apparatus for an animal that has at least two legs. The apparatus comprises a first tubular member adapted to fit over one of the animal's legs, a second tubular member adapted to fit over another of the animal's legs, and a support member connected to the first tubular member and the second tubular member. The support member is adapted to fit over the animal's back. The present invention also provides a method of making a bandage for an animal. The method comprises the steps of providing a first tubular member adapted to fit over one of the animal's legs, providing a second tubular member adapted to fit over another of the animal's legs, and providing a support member that is adapted to be connected to the first tubular member and the second tubular member. The support member adapted to fit over the animal's back.

An example of the present invention is it use for an animal that has hurt its arm (leg). For example, the animal has an injury on its elbow. Prior to the present invention, it was necessary to continually re-wrap the wound with tape. The cast/tape would not stay over the animal's elbow. Using the present invention the animal's cut is covered with a medicated pad. Then the medicated pad is covered with the tubular member of the bandage system of the present invention to keep the wound and medicated pad clean and in place. The bandage system can be made with two cut tubular members and elastic over the animal's back. The system worked unexpectedly well. The two tubular members remained in place and protected the medication that had been applied to the animal's wound. In one embodiment, the present invention provides a bandage apparatus for a dog that has a first front leg, a second front leg, two back legs, and a back. The apparatus of this embodiment is specifically, a first tubular member made of fabric, said first tubular member adapted to fit over the dog's first front leg, a second tubular member made of fabric, said second tubular member adapted to fit over the dog's second front leg, and an adjustable strap attached to the first tubular member and the second tubular member, the adjustable strap is adapted to fit over the dog's back. Prior to the present invention, it was necessary to continually re-wrap the dog's wound with tape. The cast/tape would not stay over the dog's elbow. Using the present invention the dogs's wound is covered with a medicated pad. Then the medicated pad is covered with the tubular member of the bandage system of the present invention to keep the wound and medicated pad clean and in place.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 is a side view of a dog with an apparatus constructed in accordance with the present invention positioned on the dog.

FIG. 2 is a front view of the dog shown in FIG.1 with an apparatus constructed in accordance with the present invention positioned on the dog.

FIGS. 3, 4, and 5 illustrate additional details of the system.

FIGS. 6, 7A and 7B, illustrate another embodiment of a system of the present invention

### DETAILED DESCRIPTION OF THE INVENTION

Referring to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

Described below are examples of the present invention as it is use for a dog that has hurt its arm. The dog has an injury on its elbow. Prior to the present invention, it was necessary to continually re-wrap the wound with tape. The cast/tape would not stay over the dog's elbow. The bandage system was made with two tubular members and elastic over the dog's back. The system worked unexpectedly well. The two tubular members remained in place and protected the medication that had been applied to the dog's wound.

Using the present invention the dog's cut was covered with a medicated pad. Then the medicated pad was covered the pad with the bandage system of the present invention to keep the wound and medicated pad clean and in place. When the dog was taken to a veterinarian, the veterinarian and the veterinarian's staff thought it was a great idea. They had not seen or heard about such a system and the system works great. They indicated that a lot of pets hurt their legs and need something to make it more comfortable for them and make it easier to deal with for the owner . . . such as not having to tape and re-tape the leg every 15 minutes.

Problems with prior art bandages for animals such as dogs are described in U.S. Pat. No. 3,895,628 issued Jul. 22, 1975 to Georga Ann Adair for a body encompassing bandage protector for dogs and other such animals and in United States Published Patent Application No. 2004/0031448 by Diana M. Trepanier published Feb. 19, 2004 for a post-surgical care bandage cover for pets. The problems are described as follows:

“Dogs and other animals have a tendency to bite and pull bandage coverings from body wounds. When the wound, whether it be a result of surgery or an accident, is unprotected by a bandage covering the healing process is likely to be slowed and the open wound is susceptible to infection. If the wound is infected the life of the animal is often placed in jeopardy and the recovery period is prolonged.”

“It would be desirable, to provide a cover for a pet which is easily mounted and removed and which can effectively cover a dressed or undressed wound, protecting the area from constant washing, and keeping the wound clean, allowing air flow but excluding dust and dirt. It would also be desirable to provide such a cover which provides easy access to the wound for inspection by a veterinarian without entirely removing the cover, a task which may be laborious and time consuming, depending on the size and disposition of the pet, while assuring that the pet cannot remove the cover for access to the wound. It would also be desirable to provide a cover which is durable, machine washable, stylish, amenable to decoration, available in a variety of colors and sizes while remaining affordable for any dog owner.”

The disclosures of U.S. Pat. No. 3,895,628 issued Jul. 22, 1975 to Georga Ann Adair for a body encompassing bandage protector for dogs and other such animals and in United States Published Patent Application No. 2004/0031448 by Diana M. Trepanier published Feb. 19, 2004 for a post-surgical care bandage cover for pets are incorporated herein by this reference.

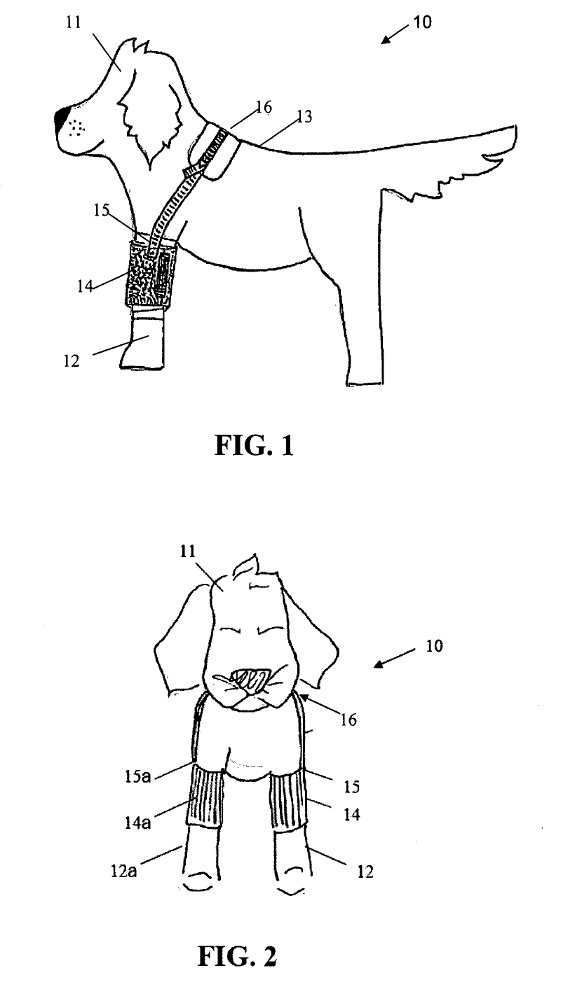

Referring now to the drawings and in particular to FIGS. 1 and 2 of the drawings, one embodiment of a bandage system for animals constructed in accordance with the present invention is illustrated. This embodiment of the present invention is designated generally by the reference numeral 10. FIG. 1 is a side view of a dog 11 with the system 10 positioned on the dog. FIG. 2 is a front view of the dog 11 shown in FIG.1 with the system 10 positioned on the dog.

The dog 11 had hurt one of her legs, leg 12. The dog 11 had an open wound on her leg 12. Efforts to treat the wound required re-wrapping the wound with tape, but the cast/tape would never stay over the dog' leg 12. Applicants made the system 10 with two tubular members 14 and 14A and an elastic strap that would fit over the dog's 11 back 13. The elastic strap can be a single strap or a strap made up of sections 15 and 15a that will fit over the dog's 11 back 13. In other embodiments, the elastic strap can have attachments that allow the strap to be connected to the two tubular members 14 and 14A. The attachments can be Velcro attachments, snaps, clips or other types of attachments. A pad 16 is positioned on the dog's back and the strap can be supported by the pad 16.

The system 10 is a system for making a bandage for the dog 11 that has a first leg 12, a second leg 12A, and a back 13. The system comprises providing a first tubular member 14 adapted to fit over the animal's first leg 14, providing a second tubular member 14A adapted to fit over the animal's second leg 12A, and providing a support member 15/15a that is adapted to be connected to the first tubular member 14 and the second tubular member 14A. The support member 15/15a is adapted to fit over the dog's 11 back 13.

The strap 15/15a is made of elastic and is flexible which prevents the tubular members 14 and 14A from moving out of place or falling off and keeps the wound covered. The strap 15/15a is adjustable which also helps prevents the tubular members 14 and 14A from moving out of place. The tubular members 14 and 14A are made of fabric. In one embodiment the tubular members 14 and 14A are sweatbands which hold the sterile gauze in place on the animal 11. In another embodiment, the tubular members 14 and 14A are made of a Velcro stretchy band material. In yet another embodiment, the tubular members 14 and 14A are made of socks that have been cut to provide the tubular members 14 and 14A. Use of socks allows easily obtainable fabric tubular members 14 and 14A to be purchased and made.

In operation the dog's 11 wound is covered with a medicated pad. The system 10 is then placed over the medicated pad to keep it clean and in place. The first tubular member 14 is positioned over the dog's first leg 14. The second tubular member 14A is positioned over the dog's 11 second leg 12A. The support member 15/15a is fits over the dog's 11 back 13. In one embodiment the pad 16 provides support for the strap 15/15a. In other embodiments, the support member 15/15a can be removably connected to the first tubular member 14 by detachable attachment and the support member 15/15a can be removably connected to the second tubular member 14A by detachable attachment.

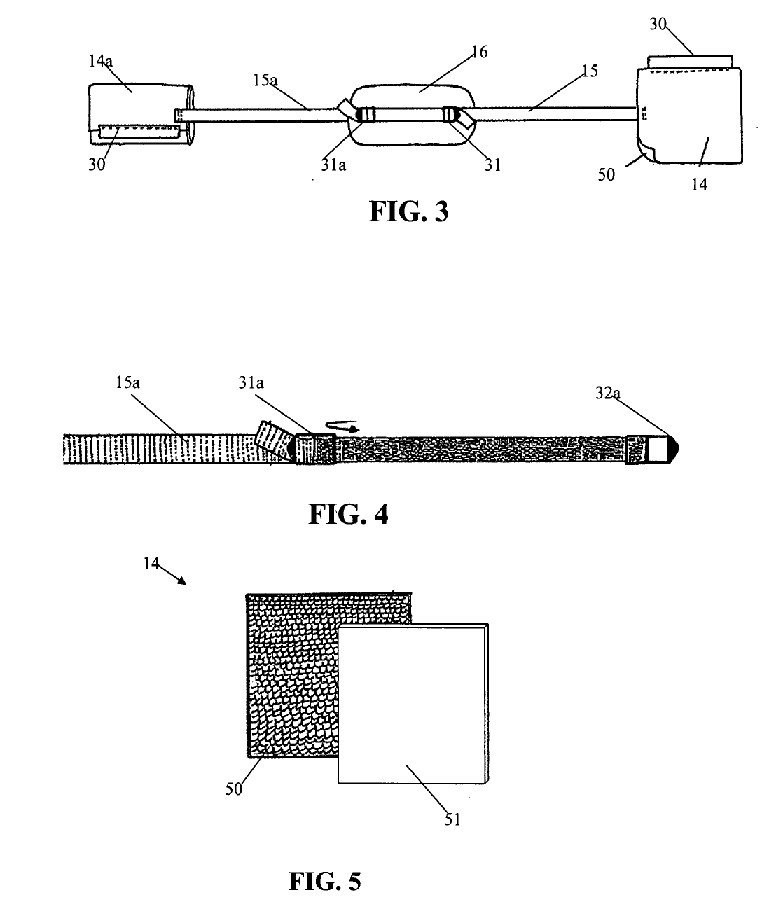

Referring now to FIGS. 3, 4, and 5 additional details of the system 10 are illustrated. Referring specifically to FIG. 4, the two tubular members 14 and 14A, the pad 6, and the elastic strap 15/15a that fits over the dog's back are illustrated. The elastic strap 15/15a is connected to the two tubular members 14 and 14A. For example the elastic strap 15/15a can connected to the two tubular members 14 and 14A by stitching. In other embodiments the elastic strap 15/15a can connected to the two tubular members 14 and 14A by attachments 31 and 31A that allow the strap 15/15a to be detachably connected to the two tubular members 14 and 14A.

The two tubular members 14 and 14A open to allow them to be place over the dog's first leg 14 and the dog's 11 second leg 12A. This is illustrated in FIG. 3. The tubular member 14a is shown closed with a Velcro closure piece 30 used to close the tubular member 14a. The tubular member 14 is shown in the open position with the Velcro closure piece 30 in the open position.

Referring now to FIG. 4 one of the portions 15a of the support member 15/15a is shown in greater detail. A plastic adjuster 31a allows the strap portion 15a to be adjusted. The strap portions 15 and 15a are used to keep the tubular members in place by being positioned over an animal's back and attached to the two tubular members 14 and 14a. The strap 15a has nylon webbing centered on a nylon covered base to hold the plastic adjuster 31a. The plastic adjuster 31a allows the strap 15a to loop through the plastic adjuster 31a and hold the system 10 in place. The strap 15a loops through the plastic adjuster 31a. In another embodiment, a connector 32a allows the strap portions 15a an15 to be detachably connected. The strap portions 15a an15 can be connected to the pad 16.

Referring now to FIG. 5, additional details of the tubular member 14 are shown. The tubular member 14 is in the open position. The tubular member 14 is made of a terrycloth section 50 and a neoprene section 51. The neoprene section 51 covers the terrycloth section 50. This provides a soft inner section 50 and a protective outer section 51. The tubular member 14 is adjustable with the Velcro attachment.

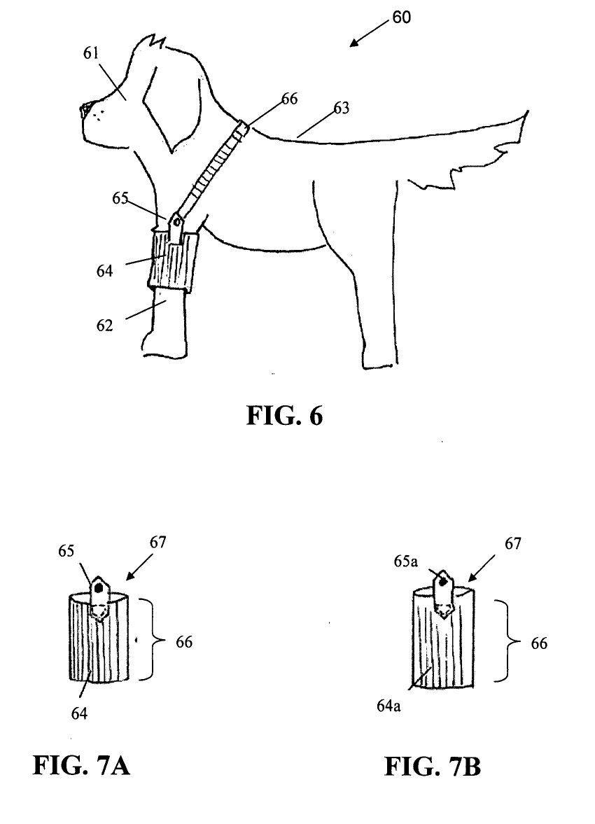

Referring now to FIGS. 6, 7A and 7B, another embodiment of a system of the present invention is illustrated. The system is designated generally by the reference numeral 60. The system 60 provides a bandage for an animal that has at least two legs. FIG. 6 is a side view of a dog 61 with the system 60 positioned on the dog. The dog 61 had hurt one of her legs, leg 62. The dog 61 had an open wound on her leg 62. Efforts to treat the wound required re-wrapping the wound with tape, but the cast/tape would never stay over the dog' leg 62. Applicant made the system 60 with two cut off socks 64 and an elastic strap 66 over the dog's back 63. The elastic strap 66 has attachments 65 that allow the strap 66 to be detachably connected to the two cut off socks 64. The attachments can be snaps, Velcro, clips, or other types of attachments.

The system 60 is a system for making a bandage for the dog 61 that has a first leg 2, a second leg, and a back 63. The system comprises providing a first tubular member 64 adapted to fit over the animal's first leg 62, providing a second tubular member adapted to fit over the animal's second leg, and providing a support member 66 that is adapted to be connected to the first tubular member 64 and the second tubular member. The support member 66 is adapted to fit over the dog's back 63.

The strap 66 is made of elastic and is flexible which prevents the tubular members from moving out of place or falling off and keeps the wound covered. The strap 66 is adjustable which also helps prevents the tubular members from moving out of place. The tubular members are made of fabric. In one embodiment the tubular members are socks that have been cut to provide the tubular members. Use of socks allows easily obtainable fabric tubular members to be purchased and made.

In operation the dog's 61 wound is covered with a medicated pad. The system 60 is then placed over the medicated pad to keep it clean and in place. The first tubular member 64 is positioned over the dog's first leg 62. The second tubular member is positioned over the dog's 61 second leg. The support member 66 is connected to the first tubular member 64 by the detachable attachment 65. The support member 66 is connected to the second tubular member by a detachable attachment. The support member 66 is fits over the dog's back 63.

Referring now to FIGS. 7A and 7B, the first tubular member 64 and the second tubular member 64a are shown. The length 66 of each tubular member 40a and 40b is substantially equal to the circumference 67 of the tubular member. The first tubular member 64 and the second tubular member 64a can be used for animals such as a dog. The first tubular member 64 and the second tubular member 64a have snaps 65 and 65a for attaching the first tubular member and the second tubular member to a strap such as the strap 66 shown in FIG. 6. The tubular members are made of fabric. In this embodiment the tubular members are socks that have been cut to provide the tubular members. Use of socks allows easily obtainable fabric tubular members to be purchased and made.

In operation the animal's wound is covered with a medicated pad. The tubular members 64 and 64a are then placed over the medicated pad to keep it clean and in place. The first tubular member is positioned over the animal's first leg. The second tubular member is positioned over the animal's second leg. The support member is connected to the first tubular member by the detachable attachment. The support member is connected to the second tubular member by the detachable attachment. The support member is placed over the animal's back.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

The invention claimed is:

1. A bandage apparatus for an animal that has at least a first leg, a second leg, and a back, comprising:
a first tubular member adapted to fit over the animal's first leg,
a second tubular member adapted to fit over the animal's second leg, and
a support member connected to said first tubular member and said second tubular member, said support member adapted to fit over the animal's back.

2. The bandage apparatus for an animal of claim 1 wherein said support member comprises an adjustable strap adapted to fit over the animal's back.

3. The bandage apparatus for an animal of claim 1 wherein said support member comprises an adjustable strap adapted to fit over the animal's back and adapted to be removeably attached to said first tubular member and said second tubular member.

4. The bandage apparatus for an animal of claim 1 wherein said first tubular member adapted to fit over the animal's first leg and said second tubular member adapted to fit over the animal's second leg comprise tubular members made of fabric.

5. The bandage apparatus for an animal of claim 1 wherein said first tubular member adapted to fit over the animal's first leg and said second tubular member adapted to fit over the animal's second leg comprise socks that have been cut to provide said tubular members.

6. The bandage apparatus for an animal of claim 1 wherein said first tubular member adapted to fit over the animal's first leg and said second tubular member adapted to fit over the animal's second leg have a length and a circumference and wherein said length is substantially the same as said circumference.

7. A method of making a bandage for an animal that has at least a first leg, a second leg, and a back, comprising the steps of:
providing a first tubular member adapted to fit over the animal's first leg,
providing a second tubular member adapted to fit over the animal's second leg, and
providing a support member that is adapted to be connected to said first tubular member and said second tubular member, said support member adapted to fit over the animal's back.

8. The method of making a bandage for an animal of claim 7 wherein said step of providing a support member comprises providing an adjustable strap adapted to fit over the animal's back.

9. The method of making a bandage for an animal of claim 7 wherein said step of providing a support member comprises providing an adjustable strap adapted to fit over the animal's back and adapted to be removeably attached to said first tubular member and said second tubular member.

10. The method of making a bandage for an animal of claim 7 wherein said steps of providing a first tubular member adapted to fit over the animal's first leg and providing a second tubular member adapted to fit over the animal's second leg comprise providing tubular members made of fabric.

11. The method of making a bandage for an animal of claim 7 wherein said step of providing a first tubular member adapted to fit over the animal's first leg comprises providing a piece of fabric with a Velcro attachment and wherein said step of providing a second tubular member adapted to fit over the animal's second leg comprises providing a piece of fabric with a Velcro attachment.

12. The method of making a bandage for an animal of claim 7 wherein said steps of providing a first tubular member adapted to fit over the animal's first leg and providing a second tubular member adapted to fit over the animal's second leg comprise comprises cutting a first sock to provide said first tubular member and cutting a second sock to provide said second tubular member.

13. A bandage apparatus for a dog that has a first front leg, a second front leg, two back legs, and a back, consisting of:
a first tubular member made of fabric, said first tubular member adapted to fit over the dog's first front leg,
a second tubular member made of fabric, said second tubular member adapted to fit over the dog's second front leg, and
an adjustable strap attached to said first tubular member and said second tubular member, said adjustable strap adapted to fit over the dog's back.

14. The bandage apparatus for a dog of claim 13 wherein said first tubular member and said second tubular member are rectangular sections made of fabric with Velcro closure pieces.

15. The bandage apparatus for a dog of claim 13 wherein said first tubular member and said second tubular member are rectangular sections made of a terrycloth section and a neoprene section.

16. The bandage apparatus for a dog of claim 13 wherein said first tubular member and said second tubular member are rectangular sections and each rectangular section has a length and the length is substantially equal to the circumference of the tubular member.

17. The bandage apparatus for a dog of claim 13 wherein said first tubular member and said second tubular member are socks that have been cut to provide said tubular members.
